(19) United States
(12) Patent Application Publication
(10) Pub. No.: US 2022/0204946 A1
(43) Pub. Date: Jun. 30, 2022

(54) MODIFIED ENVELOPE GLYCOPROTEINS FOR RETROVIRIDAE VIRAL VECTOR PSEUDOTYPING AND PROCESS FOR OBTAINING IT

(71) Applicant: INSTITUTO DE BIOLOGIA EXPERIMENTAL E TECHNOLÓGICA (IBET), Oeiras (PT)

(72) Inventors: Hélio ANTUNES TOMÁS, Torres Vedras (PT); Daniel Alexandre FERNANDES MESTRE, Vila Real De Santo António (PT); Ana Filipa DE ALBUQUERQUE FERREIRA RODRIGUES, Oeiras (PT); Ana Sofia DE SOUSA VALENTE COROADINHA, Lisboa (PT)

(21) Appl. No.: 17/612,057

(22) PCT Filed: May 20, 2020
(86) PCT No.: PCT/IB2020/054804

§ 371 Date: Nov. 17, 2021

(30) Foreign Application Priority Data
May 20, 2019 (PT) 115529

(51) Int. Cl. C12N 7/00; C12N 15/86

(52) U.S. Cl. CPC C12N 7/00; C12N 15/86; C12N 2740/15043; C12N 2740/13052; C12N 2740/15051; C12N 2740/13022

(57) ABSTRACT

The present invention describes the development of a modified envelope glycoproteins to pseudo-type viruses of the retroviridae family. These are derived from Murine leukaemia virus amphotropic, Gibbon Ape leukaemia virus and feline endogenous virus envelopes.

The improved envelope glycoproteins contain, among other modifications, newly introduced alternative cleavable sequences.

The viral vectors pseudo-typed with these modified envelopes may be suitably employed for cargo delivery such as in gene and cell therapy applications, for the ex vivo and in vivo delivery of gene(s), protein(s), or molecule(s) of interest to a variety of target cells.

### TECHNICAL DOMAIN OF THE INVENTION

The present invention relates to the development of modified envelope glycoproteins to pseudo-type viruses of the retroviridae family. These are derived from Murine leukaemia virus amphotropic, Gibbon Ape leukaemia virus and feline endogenous virus envelopes. The improved envelope glycoproteins contain, among other modifications, newly introduced alternative cleavable sequences.

The viral vectors pseudo-typed with these modified envelopes may be suitably employed for cargo delivery such as in gene and cell therapy applications, for the ex vivo and in vivo delivery of gene(s), protein(s), or molecule(s) of interest to a variety of target cells.

The modified envelope glycoproteins to pseudo-type viruses can be utilised using transient co-transfection of plasmids system or to develop stable cell lines producing recombinant viruses.

Therefore, the present invention is in the area of genetic engineering, diagnose, pharmaceutic and medical to be applied in medical human healthcare such as in gene and cell therapy applications, for the ex vivo and in vivo delivery of gene(s), protein(s), or molecule(s) of interest to a variety of target cells. Notwithstanding, it can be also used as a research tool or for veterinary and other applications.

### BACKGROUND OF THE INVENTION

The use of a highly pathogenic human virus for cargo delivery in therapeutic applications raises serious biosafety concerns. Therefore, the design of packaging systems evolved in order to increment the efficiency and the safety of retroviridae vectors while minimizing the possibility of replication-competent viruses during vector production.

Currently, several viral vectors derived from retroviridae virus family were developed to be used as therapeutics. Belonging to different genus of retroviridae are Alpharetrovirus, Betaretrovirus, Gammaretrovirus, Deltaretrovirus, Epsilonretrovirus, Lentivirus and Spumavirus. From the later, lentivirus derived vectors are currently the ones presenting the highest rate of utilization in clinical trials and have been approved to be used in the clinic.

Four generations of lentiviral vectors are considered. The first generation, developed by Naldini and co-workers, (Naldini, L. et al. In vivo gene delivery and stable transduction of nondividing cells by a lentiviral vector. Science 272, 263-7 (1996)) consisted in a three expression cassettes system. The packaging cassette had all structural, accessory and regulatory proteins, with the exception of the envelope glycoprotein. The transgene cassette was composed by the 5′LTR, the packaging signal, the RRE cis-acting region and the transgene under the control of a heterologous promoter. In the envelope glycoprotein expression cassette, the native HIV-1 envelope glycoprotein was replaced by the vesicular stomatitis virus G glycoprotein (VSV-G). With this system, good titers were easily achieved, but the poor safety level could not be accepted for a human and potentially lethal pathogen. Replication competent lentiviruses (RCL) could be generated with three homologous recombination events between the viral sequences of the packaging and transgene cassette or endogenous retroviral sequences within the producer cells. Additionally, the presence of the LTR promoter could activate neighbouring cellular genes, and the presence of all accessory HIV-1 genes, which are incorporated into the viral particle, enhanced the immunogenicity of the vector.

In the second generation, all accessory genes were deleted from the three plasmid system, without negative effects on vector titer. By removing the accessory genes (vpr, vif, vpu and nef), the generation of RCL became less probable and, if generated, would be unlikely to be pathogenic.

The third and still most widely used generation was developed by Dull and co-workers (Dull, T. et al. A third-generation lentivirus vector with a conditional packaging system. J. Virol. 72, 8463-71 (1998)). It is characterized by the deletion of the tat gene from the packaging plasmid and rev gene placed in an independent plasmid. By the use a chimeric 5′LTR with a heterologous viral promoter/enhancer, such as those of Cytomegalovirus (CMV) or Rous sarcoma virus (RSV). Therefore, lentiviral vector expression is independent of Tat. Rev is maintained but is provided by an independent non-overlapping plasmid.

Thus, this system has a total of four constructs, increasing the number of homologous recombination events required for RCL formation.

Another feature of this generation is the partial deletion of the 3′LTR in the transgene cassette, leading to transcriptional inactivation of the LTR promoter, after reverse transcription. These vectors are called self-inactivating (SIN) vectors.

This inactivation increases safety and reduces concerns related to insertional mutagenesis in the neighbouring sequences that can lead to the transactivation or up-regulation of neighbouring genome sequences, such as oncogenes.

A fourth generation of lentiviral vectors, Rev-independent, has also been developed by means of replacing RRE with heterologous viral sequences or by codon-optimization. These packaging systems are not, however, easily available for the research community. Also, the reported titers are one to two logs lower than that of the second or third generation systems.

Lentiviral vectors can be produced by transient transfection or stable producer cell lines. In transient transfection production, cells are co-transfected with the viral constructs, for example in the third generation system it is necessary to provide: (1) the viral vector genome transgene plasmid, (2) gag-pol helper plasmid, (3) Rev helper plasmid and (4) the plasmid expressing the envelope glycoprotein. Between 24 to 72 hours post-transfection, the lentiviral vectors present in the supernatant are harvested.

Stable production relies on packaging cell lines (PCLs) in which all of the components necessary to produce vectors are integrated into the cells' genome. In the scope of the present invention also semi-stable productions were performed. In this procedure only one, two or three of the above mentioned plasmids are stably integrated in the cell and therefore the productions of lentiviral vectors is performed by transiently transfecting these cells with three, two or one of the remaining necessary plasmids respectively.

The envelope glycoprotein used to pseudo-type the viral vectors defines the tropism of the virus by interacting with specific cell surface proteins and promoting the entrance of viruses into the host cell. The natural tropism of HIV-1 envelope glycoprotein is restricted to CD4+ cells, thus limiting its gene therapy applications to CD4+ cells like macrophages or T cells. However, lentiviral vectors have the ability to incorporate in their viral particles envelope glycoproteins from other viruses. This feature, denominated pseudo-typing, allows the manipulation of vector tropism.

The most used envelope glycoprotein for pseudo-typing lentiviral vectors is VSV-G, due to its wide tropism (possibly pantropism), high titres provided and improved vector stability, allowing the concentration of the particles by ultracentrifugation and resistance to freeze-thaw cycles. Despite all the advantages, VSV-G is toxic to producer cells, posing a challenge for stable production of lentiviral vectors pseudo-typed with this envelope glycoprotein.

Moreover, the broad tropism of VSV-G can be an impediment for targeted transduction of specific tissues, for example, for in vivo applications. Another limitation to its use for in vivo application is the inactivation of VSV-G by human complement present in the blood.

Several alternative envelope glycoproteins have been studied and are also suitable for pseudo-typing lentiviral vectors, for example, the amphotropic Murine leukaemia virus (MLV) 4070A envelope glycoprotein which is able to transduce most cells.

Other envelope glycoproteins have been engineered to pseudo-type lentiviral vectors with increased efficiency, for example the chimeric envelope glycoproteins RD114A and RDpro derived from the endogenous Feline leukaemia virus (RD114) and GaLV10A1 derived from the Gibbon ape leukaemia virus (GaLV).

Despite specific advantages and disadvantages, each envelope glycoprotein confers a different set of properties to the lentiviral vector, and so each pseudo-type may have its own potential niche.

The present invention aims to develop a tool, in particular an envelope glycoprotein for retroviridae viral vector pseudo-typing (including lentiviral vectors) that allows to overcome the problems mentioned above of the prior art by enhancing viral titres by modifying the cleavage site of the HIV-1 protease in the TM subunit resulting in a product with reduced toxicity when compared to VSV-G envelope.

### SUMMARY OF THE INVENTION

The present invention relates to the development of a modified envelope glycoproteins to pseudo-type viruses of the retroviridae family. These are derived from Murine leukaemia virus amphotropic, Gibbon Ape leukaemia virus and feline endogenous virus envelopes. The improved envelope glycoproteins contain, among other modifications, newly introduced alternative cleavable sequences.

For this purpose, mutant Gammaretroviruses envelope glycoproteins based on 4070A, RD114A and GaLV10A1 are described and in order to enhance viral titers the R peptide region and or cleavage site of the HIV-1 protease in the TM subunit were engineered.

In addition to wild type lentiviral protease a mutated version of the HIV-1 protease is also described. This mutation is referred as leading to a 5- to 10-fold decrease in the protease activity compared to the WT HIV-1 protease and an expected, reduced cytotoxicity without effecting virus maturation and infectivity.

In summary novel chimeric envelope glycoproteins were engineered at the cytoplasmatic tail. All envelope variants developed showed to be incorporated in retroviral particles being suitable for viral vector pseudotyping and thus in cargo delivery. The envelope proteins modified from 4070A and RD114A can be used alternatively to the original counterparts to provide higher titers when using less active retroviral proteases. The novel GaLV10A1 derived glycoproteins can be used to improve viral titers when using WT or less active viral proteases such as T26S viral protein.

The increase in virus yields obtained, reduces the required amount of material required for clinical applications, and therefore reduce its costs and the complexity of such procedures.

### DESCRIPTION OF THE FIGURES

FIG. 1. Schematic Representation of the Plasmids Used in Lentiviral Vector Production.

A) Expression vectors for lentiviral genome, Gag-Pro-Pol, Rev and VSV-G envelope expression (from top to bottom).

B) Expression vectors developed for 4070A envelope and their derivatives.

C) Expression vectors developed for RD114A and their derivatives.

D) Expression vectors developed for GalVA and their derivatives.

Abbreviations: CMV, Cytomegalovirus promoter; RSV, Rous Sarcoma Virus promoter; hPGK, human phosphoglycerate kinase promoter; Int, intron; GFP, green fluorescence protein; GPP, gag-pro-pol sequence; GP(T26S)P, gag-pro-pol with mutated T26S protease sequence; Ψ, packaging signal sequence; WPRE, woodchuck hepatitis posttranscriptional regulatory element; pAn, polyA sequence; ZeoR, zeocin resistance gene; VSV-G, glycoprotein G of the vesicular stomatitis virus; resistance gene.

FIG. 2. Schematic Representation of Engineered Envelope Glycoproteins.

On top are represented the surface and transmembrane subunits and domains of the envelope glycoproteins: ectodomain, transmembrane domain (TMD) and cytoplasmic tail (CT).

The blue, sky blue, green and orange bars represent sequences from 4070A, 10A1, RD114 and GaLV envelope glycoproteins, respectively.

The square outlined in black is the R-peptide sequence.

The white, yellow and red lightning bolt shapes represent the non-modified, pro and giflet mutations on the protease cleavage site of the R-peptide, respectively. *: original envelope glycoproteins.

FIG. 3. Representative Pictures of HEK 293T Cells Transiently Expressing Envelope Glycoproteins.

Representative pictures taken during transient transfection of 293T cells with the envelope glycoproteins 4070A, RD114 and GaLV and their derivatives as indicated in the photographs legends. 80× Bright field microscopy. Scale bar: 45 μm.

FIG. 4. Lentiviral Vector Production with WT or T26S HIV-1 Protease and Engineered Envelope Glycoproteins.

The bars correspond to infectious particles and the dots to total particles. The numbers on the top of the bars indicate fold increase of infectious titer relatively to the corresponding non modified envelope glycoprotein.

All values are shown as average±standard deviation of three biological replicates (n=3). Fold increase is shown for significant changes based on a one-tailed non-paired t-test, *p<0.1 and **p<0.01. Detection limit of 4.0×10⁴ I.P./ml is indicated by a dashed arrow. WT: wild-type HIV-1 protease; T26S: T26S HIV-1 protease.

### DESCRIPTION OF THE INVENTION

The present invention relates to the development of modified envelope glycoproteins to pseudo-type viruses of the retroviridae family and to the obtained envelope glycoproteins. The improved envelope glycoproteins contain, among other modifications, newly introduced alternative cleavable sequences.

The viral vectors pseudo-typed with these modified envelopes may be suitably employed for cargo delivery such as in gene and cell therapy applications, for the ex vivo and in vivo delivery of gene(s), protein(s), or molecule(s) of interest to a variety of target cells.

1. Process for Generating Modified Envelope Glycoproteins

The modified envelope glycoproteins to pseudo-type viruses of the retroviridae family are derived from Murine leukaemia virus amphotropic, Gibbon Ape leukaemia virus and feline endogenous virus envelopes. The improved envelope glycoproteins contain, among other modifications, newly introduced alternative cleavable sequences and can be utilised using transient co-transfection of plasmids system or to develop stable cell lines producing recombinant viruses.

Therefore, mutant Gammaretroviruses envelope glycoproteins based on 4070A, RD114A and GaLV10A1 are described and in order to enhance viral titres the R peptide region and or cleavage site of the HIV-1 protease in the TM subunit were engineered as described below.

2. Lentiviral Vectors Production

Lentiviral vectors can be produced by transient transfection or stable producer cell lines. In transient transfection production, cells are co-transfected with the viral constructs, for example in the third generation system it is necessary to provide: (1) the viral vector genome transgene plasmid, (2) gag-pol helper plasmid, (3) Rev helper plasmid and (4) the plasmid expressing the envelope glycoprotein. Between 24 to 72 hours post-transfection, the lentiviral vectors present in the supernatant are harvested.

In stable cell lines all the plasmids above are stably integrated in a mammalian cell line generating a producer cell which produces continuously lentiviral vectors.

In addition, two stable and transient production lentiviral vectors can be produced in semi-stable mode. In the latter case one (up to three) constructs mentioned above are stably integrated and productions can be carried out by providing transiently the remaining constructs.

2.1 Lentiviral Vectors Production by Transient Transfection

The generation of the constructs codifying the envelopes are described below.
I. pCMV-GaLV10A1 is constructed by removing a 19 nucleotides sequence prior to the start codon of GaLV10A1 from phGaLV10A1 by inverse PCR. phGaLV10A1 encodes GaLV10A1 and zeocin resistance marker under the transcriptional control of the CMV promoter and contains rabbit beta-globin (RBG) and hemoglobin subunit beta-2 (HBB2) introns upstream the start codon. GaLV10A1 is a modified GaLV envelope glycoprotein with the substitution of the cytoplasmic tail by that of the MLV clone 10A1, as described in Stitz, J. et al. (Stitz et al. Lentiviral vectors pseudotyped with envelope glycoproteins derived from gibbon ape leukaemia virus and murine leukaemia virus 10A1. Virology 273, 16-20. 2000).
II. pCMV-4070A and pCMV-RD114A encode envelope glycoprotein 4070A of the amphotropic MLV and a modified RD114 envelope glycoprotein, RD114A, as described by Sandrin, V. et al. (Sandrin, V. et al. Lentiviral vectors pseudotyped with a modified RD114 envelope glycoprotein show increased stability in sera and augmented transduction of primary lymphocytes and CD34+ cells derived from human and nonhuman primates. Blood 100, 823-32. 2002) and Zhang, X. Y. et al. (Zhang, X. Y., La Russa, V. F. & Reiser, J. Transduction of bone-marrow-derived mesenchymal stem cells by using lentivirus vectors pseudotyped with modified RD114 envelope glycoproteins. J. Virol. 78, 1219-29. 2004), with the substitution of the cytoplasmic tail by that of the 4070A, respectively.
   pCMV-4070A and pCMV-RD114A are derived from phGaLV10A1 in which GaLV10A1 sequence is removed by EcoRI and KasI restriction and replaced by 4070A and RD114A, respectively. 4070A and RD114A were amplified by PCR from pMonoZeo-4070A, as described by Tomás H A et al. (Tomás HA, Rodrigues A F, Carrondo M J T, Coroadinha A S. LentiPro26: novel stable cell lines for constitutive lentiviral vector production. Sci Rep. 8(1):5271. 2018) and pLTR-RD114A, as described by Zhang, X. Y. et al. (Zhang, X. Y., La Russa, V. F. & Reiser, J. Transduction of bone-marrow-derived mesenchymal stem cells by using lentivirus vectors pseudotyped with modified RD114 envelope glycoproteins. J. Virol. 78, 1219-29. 2004).
III. pCMV-4070AΔR, pCMV-GaLV10A1ΔR and pCMV-RD114AΔR encode 4070A, GaLV10A1 and RD114A, respectively, with the deletion of the R-peptide from the cytoplasmic tail. Each plasmid is amplified by inverse PCR from the parental plasmids previously described, to remove the nucleotides coding for the R-peptide of the cytoplasmic tail of the envelope glycoprotein genes.
IV. pCMV-4070Apro, pCMV-GaLV10A1pro and pCMV-RD114Apro encode 4070A, GaLV10A1 and RD114A, respectively, in which the R-peptide cleavage site sequence was replaced by that of the HIV-1 matrix/capsid (MA/CA) in HIV-1 Gag. Each plasmid was amplified by inverse PCR from the parental plasmids to substitute the natural R-peptide cleavage site sequence—VQAL↓VLTQ (amino acid sequence)—with the cleavage site of MA/CA-SQNY↓PIVQ.
V. pCMV-4070Agiflet, pCMV-GaLV10A1giflet and pCMV-RD114Agiflet encode 4070A, GaLV10A1 and RD114A, respectively, with a synthetic R-peptide cleavage site sequence reported as the most efficiently cleaved peptide site GSGIF↓LETSL by the HIV-1 protease, as described by Beck, Z. Q. et al. (Beck, Z. Q., Hervio, L., Dawson, P. E., Elder, J. H. & Madison, E. L. Identification of efficiently cleaved substrates for HIV-1 protease using a phage display library and use in inhibitor development. Virology 274, 391-401. 2000). The construction of these plasmids was conducted in two steps to replace the natural R-peptide cleavage site sequence with the synthetic sequence, where in the first step, each plasmid was amplified by inverse PCR from the parental plasmids to substitute the sequence VLTQ of the natural R-peptide cleavage site with the LETSL, and in the second step, the same approach was used to replace the VQAL sequence with the GSGIF sequence.

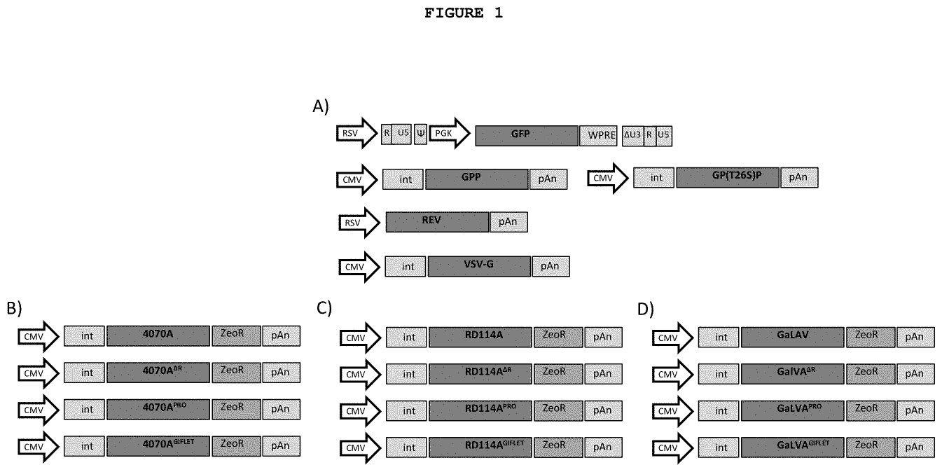

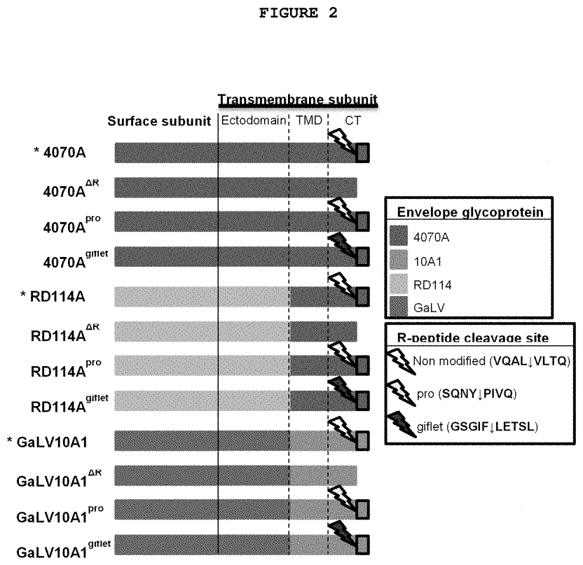

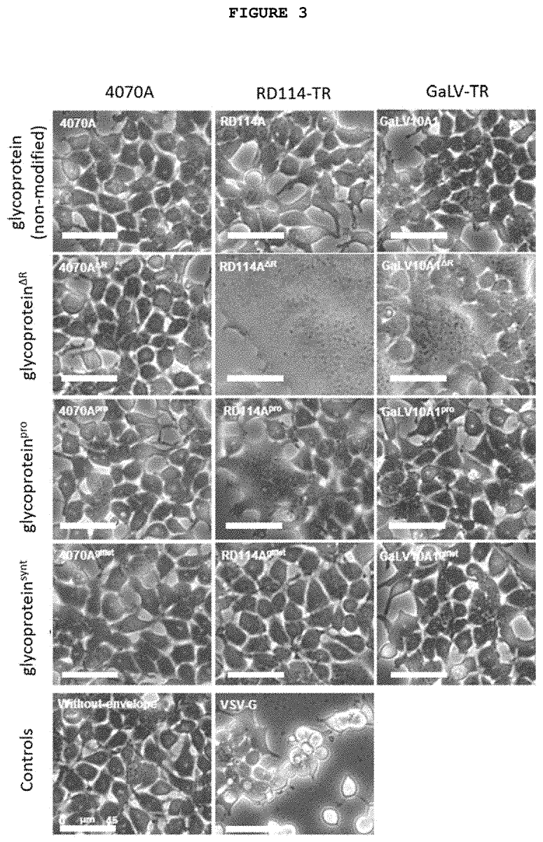

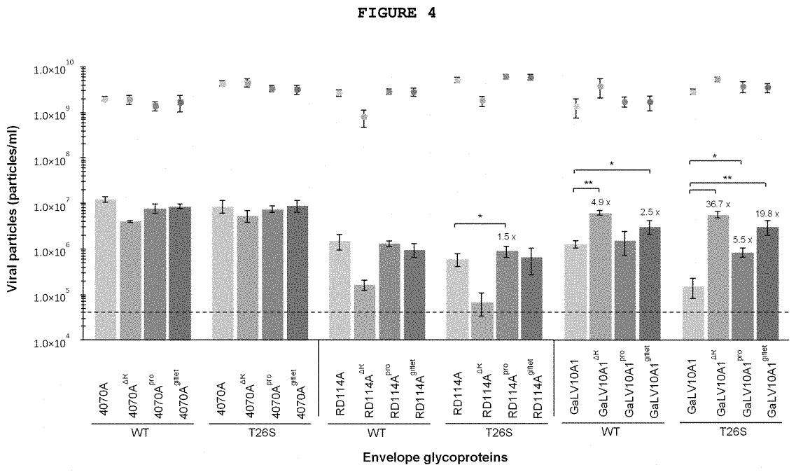

Schematic representations of the constructed plasmids main transcriptional units are provided in FIGS. 1 B, C and D.

The transient transfection production of lentiviral vectors is performed for example from a third generation lentiviral packaging system by transfection of plasmid DNA, comprising:
a viral vector genome transgene plasmid,
a gag-pol helper plasmid having packaging functions either having a WT or T26S mutation in the HIV-1 protease active site,
a Rev helper plasmid transgene vector, and
a plasmid expressing the envelope glycoprotein.

2.2 Lentiviral Vectors Production by Stable Producer Cell Lines

Stable production relies on packaging cell lines (PCLs) in which all of the components necessary to produce vectors are integrated into the cells' genome.

In short, the process of the present invention comprises the production of a Gammaretrovirus modified envelope glycoproteins for vector pseudo-typing viruses of the retroviridae family derived from Murine leukaemia virus amphotropic, Gibbon Ape leukaemia virus or feline endogenous virus envelopes by modifying the transmembrane TM unit, altering its processing (i.e. cleavage), said process comprising the production of lentiviral vectors by stable production, wherein:
the Gammaretrovirus modified envelope glycoproteins are derived from 4070A, RD114 and GaLV derived from the Murine leukaemia virus amphotropic, the endogenous Feline leukaemia virus and the Gibbon ape leukaemia virus respectively, and
the lentiviral production vectors comprise one of the following plasmids: (i) pCMV-GaLV10A1, pCMV-4070A or pCMV-RD114A, (ii) pCMV-4070AΔR, pCMV-GaLV10A1ΔR or pCMV-RD114AΔR, (iii) pCMV-4070Apro, pCMV-GaLV10A1pro or pCMV-RD114Apro, (iv) pCMV-4070Agiflet, pCMV-GaLV10A1giflet or pCMV-RD114Agiflet.

In a preferred embodiment of the invention, the stable production of lentiviral vectors is performed from for example a third generation lentiviral packaging system stably integrated in a producer cell line containing:
a viral vector genome transgene plasmid,
a gag-pol helper plasmid having packaging functions either having a WT or T26S mutation (or other mutation) in the HIV-1 protease active site,
a Rev helper plasmid transgene vector, and
a plasmid expressing the envelope glycoprotein.

In exceptional cases the production can be carried out in semi-stable productions where some, but not all, constructs can be provided in transient to a cell stably expressing the remaining constructs.

3. Modified Envelope Glycoproteins

The most used envelope glycoprotein for pseudo-typing lentiviral vectors is VSV-G, due to its wide tropism, high titres provided and improved vector stability. However, VSV-G is toxic to producer cells. Therefore, the present invention proposes an alternative modified envelope glycoprotein for pseudo-typing lentiviral vectors that provides high titres and potentially improved vector stability, allowing the concentration of the particles by ultracentrifugation and resistance to freeze-thaw cycles and is safe.

These envelope glycoproteins are based on envelope glycoproteins 4070A derived from the murine leukaemia virus, RD114A derived from the endogenous Feline leukaemia virus and GaLV10A1 derived from the Gibbon ape leukaemia virus (GaLV) but modified in order to enhance viral titres through improved processing the TM subunit.

In result of this, modified sequences from 4070A, RD114 and GaLV envelope glycoproteins were produced with mutations on the TM namely on the protease cleavage site of the R-peptide by introduction of alternative cleavable sequences.

A short sequence—R-peptide—is cleaved from the cytoplasmic tail of retroviral envelope glycoproteins. Enhanced cleavage is expected when (i) homologous cleavage sequences, in relation to the viral protease, are used and (ii) highly active proteases are employed. The influence of the protease cleavage sequence on the R-peptide cleavage site and its impact on viral particles production was evaluated.

To this end, cleavage sites specifically recognizable by the HIV-1 protease were introduced in 4070A, RD114A and GaLV10A1. These envelope glycoproteins shared the retroviral cleavage site—VQAL↓VLTQ—of the cytoplasmic tail of 4070A. Herein, several envelope glycoproteins chimeras were constructed, engineered at the protease cleavage site of the R-peptide, to contain cleavage sequences recognized by HIV-1 proteolytic processing. These new glycoproteins were compared with their counterparts harbouring a murine leukaemia virus cleavage sequence.

For each envelope glycoprotein three mutations were performed:
ΔR: the removal of the R-peptide, generating a truncated cytoplasmic tail, similar to described by Christodoulopoulos, I. & Cannon, P. M. (Christodoulopoulos, I. & Cannon, P. M. Sequences in the Cytoplasmic Tail of the Gibbon Ape Leukaemia Virus Envelope Protein That Prevent Its Incorporation into Lentivirus Vectors. J. Virol. 75, 4129-38. 2001) and Rein, A. et al. (Rein, A., Mirro, J., Haynes, J. G., Ernst, S. M. & Nagashima, K. Function of the cytoplasmic domain of a retroviral transmembrane protein: p15E-p2E cleavage activates the membrane fusion capability of the murine leukaemia virus Env protein. J. Virol. 68, 1773-81. 1994);
pro: cleavage site sequence of HIV-1 matrix/capsid (MA/CA)—SQNY↓PIVQ
giflet: synthetic cleavage site sequence reported as the most efficiently cleaved peptide site by the HIV-1 protease—GSGIF↓LETSL, as described by Beck, Z. Q. et al. (Beck, Z. Q., Hervio, L., Dawson, P. E., Elder, J. H. & Madison, E. L. Identification of efficiently cleaved substrates for HIV-1 protease using a phage display library and use in inhibitor development. Virology 274, 391-401. 2000).

Schematic representations of the constructed chimeric modified glycoproteins are provided in FIG. 2 where the surface and transmembrane subunits and domains of the envelope glycoproteins, such as the ectodomain, transmembrane domain (TMD) and cytoplasmic tail (CT) are depicted.

FIG. 4 summarizes the lentiviral vector productions results obtained for the 12 envelope glycoproteins. Mutations in the R-peptide cleavage site were found to mildly affect the infectious titers of lentiviral vectors pseudotyped with 4070A glycoprotein variants. RD114A modifications impacted the titers both negatively (RD114A^{ΔR}) and positively) (RD114A^{pro}) when compared to the original counterpart. For GaLV10A1 derived glycoproteins, all mutations increased viral titers. Remarkably, infectious particles yields improvements up to 37-fold were observed for T26S HIV protease. Also an effect was also observed for WT HIV-1 protease, with a 5-fold improvement.

4. Viral Vectors

Several viral vectors derived from retroviridae virus family were developed, in particular lentivirus derived vectors comprising the modified envelope glycoproteins based on 4070A, RD114A and GaLV10A1 according to the present invention were developed such as:
Expression vectors developed for 4070A envelope and their derivatives,
Expression vectors developed for RD114A and their derivatives, and
Expression vectors developed for GalVA and their derivatives.

FIG. 1 shows a representation of the plasmids used in lentiviral vector production.

pMDLg/pRRE^{T26S} and pMDLg/pRRE^{D25N} are in-house constructed plasmids derived from pMDLg/pRRE, with the mutations T26S and D25N in the HIV-1 protease active site, respectively. The T26S mutation is described to cause reduced proteolytic activity and loss of protease-mediated cytotoxicity; the D25N mutation inactivates the active site of the protease. Also pRRLSIN-CMV-GFP plasmid was constructed derived from pRRLSIN.cPPT.PGK-GFP.WPRE driving the expression of enhanced green fluorescent protein (eGFP) from the CMV promoter.

5. Cell Lines Expressing the Modified Envelope Glycoproteins

Cell lines expressing the different glycoproteins described previously can be obtained by stable viral vector production. Stable production relies on packaging cell lines (PCLs) in which all of the components necessary to produce vectors are integrated into the cells' genome.

FIG. 3 shows HEK 293T cells transiently expressing envelope glycoproteins where transient transfection of 293T cells with the envelope glycoproteins 4070A, RD114 and GaLV and their derivatives was performed.

### EXAMPLES

### Example 1: Process for Lentiviral Vectors Production and Titration

For transient production of lentiviral vectors, the third generation lentiviral packaging system and the transfection procedure as described in Tomás et al. (2013 and 2018) were used (Tomás, H. A., Rodrigues, A. F., Alves, P. M. Coroadinha, A. S. Lentiviral Gene Therapy Vectors: Challenges and Future Directions. Gene Therapy—Tools and Potential Applications (ed. Martin, F.). InTech (2013) pp. 287-317), (Tomás HA, Rodrigues A F, Carrondo M J T, Coroadinha A S. LentiPro26: novel stable cell lines for constitutive lentiviral vector production. Sci Rep. 8(1):5271. 2018).

The transfection procedure was conducted using PEI. HEK 293T cells were seeded at 5×10⁴ cells/cm² in 25 cm² t-flask 24 h prior to transfection. A total of 4.65 μg of plasmid DNA per million cells was used for the transfection of one t-flask: 1 μg of pMDLg/pRRE or its variants (T26S and D25N) and 0.25 μg of pRSV-Rev (providing the packaging functions), 2.5 μg of pRRLSIN-CMV-GFP (providing the transgene vector) and 0.9 μg of plasmid codifying the envelope glycoprotein. After 20 to 24 hours post transfection, the medium was replaced with 4 ml of DMEM supplemented with 10% (v/v) FBS.

To assess transfection efficiency, transfected cells were harvested and analyzed for GFP fluorescence by flow cytometry (CyFlow® Space, Sysmex Corporation, Kobe, Japan).

pMDLg/pRRE^{T26S} and pMDLg/pRRE^{D25N} are in-house constructed plasmids derived from pMDLg/pRRE, with the mutations T26S and D25N in the HIV-1 protease active site, respectively. The T26S mutation is described to cause reduced proteolytic activity and loss of protease-mediated cytotoxicity; the D25N mutation inactivates the active site of the protease.

pRRLSIN-CMV-GFP is a third generation lentiviral transgene plasmid, driving the expression of enhanced green fluorescent protein (eGFP) from the CMV promoter. This plasmid is an in-house constructed plasmid, derived from pRRLSIN.cPPT.PGK-GFP.WPRE, as described by Dull, T. et al. (Dull, T. et al. A third-generation lentivirus vector with a conditional packaging system. J. Virol. 72, 8463-71. 1998) where the human phosphoglycerate kinase 1 (PGK) promoter was replaced by the CMV promoter.

Schematic representations of the plasmids main transcriptional units are provided in FIG. 1.

Physical (total) particles (T.P.) in the serum-free viral supernatant were assessed by Nanoparticle Tracking Analysis (NTA) using NanoSight® NS500 (Malvern Instruments Ltd, Malvern, UK), following the manufacturer's instructions.

For titration of total particles by nanoparticle tracking analysis, serum-free DMEM was used in this step. After an additional production period of 24 hours, the medium containing the viral vectors was harvested, filtered through 0.45 μm-pore-size cellulose acetate filter for clarification, aliquoted and stored at −80° C. until further use.

For viral supernatants containing serum, a p24 enzyme-linked immunosorvent assay (ELISA)—INNOTEST HIV Antigen mAb (Fujirebio Diagnostics, Inc., Malvern, Pa., USA)—was used to quantify total particles, according to the manufacturer's instructions.

For titration of infectious particles (I.P.), HEK 293T cells were seeded at 5×10⁴ cells/cm² in 24-well plates 24 hours before infection. Transduction was performed by removing the cell supernatant and infecting cells with 0.2 ml of viral supernatants at several dilutions performed in DMEM supplemented with 10% (v/v) FBS containing 8 μg/ml of polybrene (Sigma-Aldrich).

For normal transduction protocol, cells were incubated at 37° C. overnight after which 0.5 ml of DMEM supplemented with 10% (v/v) was added. For spin inoculation protocol, the plates were centrifuged at 1200×g, 25° C. for 2 hours after which 0.5 ml of fresh supplemented DMEM was added and cells were incubated at 37° C. Two days after infection, cells were harvested and analyzed for GFP fluorescence by flow cytometry (CyFlow® Space).

The I.P. titer was determined taking into account the percentage of GFP positive cells, the number of cells determined at infection time and the dilution factor of the viral supernatant. Infections that rendered 2-20% of infected cells were considered for titer calculations.

### Example 2: Application of the Modified Envelope Glycoproteins for Viral Vector Pseudotyping

Gammaretrovirus envelope glycoproteins, unlike VSV-G, undergo proteolytic processing during virion assembly mediated by the retroviral protease. A short sequence—R-peptide—is cleaved from the cytoplasmic tail, as described by Tedbury, P. R. & Freed, E. O. (Tedbury, P. R. & Freed, E. O. The cytoplasmic tail of retroviral envelope glycoproteins. Prog. Mol. Biol. Transl. Sci. 129, 253-84. 2015). This cleavage is required for virus entry, since it activates the fusogenic activity of the envelope glycoprotein.

The R-peptide cleavage site in the original envelope glycoproteins is specifically recognized by the retroviral protease. The efficiency of cleavage is dependent on both the sequence of cleavage and the protease used (i.e. its virus family origin and introduced mutations). Enhanced cleavage is expected when (i) homologous cleavage sequences, in relation to the viral protease, are used and (ii) highly active proteases are employed. The influence of the protease cleavage sequence on the R-peptide cleavage site and its impact on viral particles production was evaluated. To this end, cleavage sites specifically recognizable by the HIV-1 protease were introduced in 4070A, RD114A and GaLV10A1. These envelope glycoproteins shared the retroviral cleavage site—VQAL↓VLTQ—of the cytoplasmic tail of 4070A. Herein, several envelope glycoproteins chimeras were constructed, engineered at the protease cleavage site of the R-peptide, to contain cleavage sequences recognized by HIV-1 proteolytic processing. These new glycoproteins were compared with their counterparts harbouring a murine leukaemia virus cleavage sequence.

In addition, for each envelope glycoprotein three mutations were performed:
ΔR: the removal of the R-peptide, generating a truncated cytoplasmic tail, as described by Christodoulopoulos, I. & Cannon, P. M. (Christodoulopoulos, I. & Cannon, P. M. Sequences in the Cytoplasmic Tail of the Gibbon Ape Leukaemia Virus Envelope Protein That Prevent Its Incorporation into Lentivirus Vectors. J. Virol. 75, 4129-38. 2001) and Rein, A. et al. (Rein, A., Mirro, J., Haynes, J. G., Ernst, S. M. & Nagashima, K. Function of the cytoplasmic domain of a retroviral transmembrane protein: p15E-p2E cleavage activates the membrane fusion capability of the murine leukaemia virus Env protein. J. Virol. 68, 1773-81. 1994);
pro: cleavage site sequence of HIV-1 matrix/capsid (MA/CA)—SQNY↓PIVQ;
giflet: synthetic cleavage site sequence reported as the most efficiently cleaved peptide site by the HIV-1 protease—GSGIF↓LETSL, as described by Beck, Z. Q. et al. (Beck, Z. Q., Hervio, L., Dawson, P. E., Elder, J. H. & Madison, E. L. Identification of efficiently cleaved substrates for HIV-1 protease using a phage display library and use in inhibitor development. Virology 274, 391-401. 2000).

Schematic representations of the constructed chimeric modified glycoproteins are provided in FIG. 2.

FIG. 3 shows 293T cells when transfected with the different glycoproteins described in FIG. 2. Syncytium formation induced by glycoproteins expression was evaluated in 293T cells transiently transfected with the plasmids coding for the envelope glycoproteins. 24 hours post transfection, cells were observed by phase-contrast microscopy. Syncytium and non-adherent round cells were observed in cells transfected with RD114AΔR, GaLVA1ΔR and VSV-G expression cassettes. Additionally, RD114Apro expression also led to few syncytia formation. In all other cases, no major morphological cell differences relative to the glycoprotein control (without any envelope expression) were observed. These syncytium formation could be eliminated by knockdown their receptor in 293T cells without affecting viral vector production (data not shown). This indicates the suitability of the usage of these envelopes both in transient but also in stable viral vector production (i.e. using packaging and producer cell lines).

The engineered envelope glycoproteins were evaluated in transient production of lentiviral vector using the wild type (WT) and the mutated (T26S) HIV-1 proteases. The productions were assessed for both total particles, quantified by p24 ELISA, and infectious particles.

FIG. 4 summarizes the lentiviral vector productions results obtained for the 12 envelope glycoproteins.

Mutations in the R-peptide cleavage site were found to mildly affect the infectious titers of lentiviral vectors pseudotyped with 4070A glycoprotein variants. RD114A modifications impacted the titers both negatively (RD114A^{ΔR}) and positively)(RD114A^{pro}) when compared to the original counterpart. For GaLV10A1 derived glycoproteins, all mutations increased viral titers. Remarkably, infectious particles yields improvements up to 37-fold were observed for T26S HIV protease. Also an effect was also observed for WT HIV-1 protease, with a 5-fold improvement.

1-10. (canceled)

11. A modified envelope glycoprotein for retroviridae viral vector pseudo-typing, derived from Murine leukaemia virus amphotropic, Gibbon Ape leukaemia virus and feline endogenous virus envelopes, comprising a cleavable sequence selected from the group consisting of: 4070Agiflet, GaLV10A1giflet, RD114Agiflet, 4070AΔR, GaLV10A1ΔR and RD114AΔR.

12. A viral vector comprising the modified envelope glycoprotein according to claim 11.

13. A cell expressing the modified envelope glycoprotein according to claim 11.

14. A cell expressing the modified envelope glycoprotein according to claim 11, wherein the cell comprises knockdown of SLC20A2, SLC1A5 and/or SLC20A1.

15. A process for producing a Gammaretrovirus modified envelope glycoproteins for vector pseudo-typing viruses of the retroviridae family derived from Murine leukaemia virus amphotropic, Gibbon Ape leukaemia virus or feline endogenous virus envelopes by introduction of an alternative cleavable synthetic sequence, said process comprising the production of lentiviral vectors by transient transfection, wherein:
the Gammaretrovirus modified envelope glycoproteins are 4070A and RD114A derived from the Murine leukaemia virus amphotropic and endogenous Feline leukaemia virus respectively and GaLV10A1 derived from the Gibbon ape leukaemia virus (GaLV), and
the lentiviral vectors comprise the following plasmids: pCMV-4070Agiflet, pCMV-GaLV10A1giflet and pCMV-RD114Agiflet, pCMV-4070AΔR, pCMV-GaLV10A1ΔR and pCMV-RD114AΔR.

16. The process according to claim 15, wherein by transient or semi-stable transfection or by stable production of lentiviral vectors is performed from lentiviral packaging systems using plasmid DNA, comprising:
a viral vector genome transgene plasmid,
a gag-pol helper plasmid having packaging functions with a wildtype or a modified viral protease,
a Rev helper plasmid vector, and
a plasmid expressing the envelope glycoprotein.

17. The process according to claim 16, wherein the modified viral protease is HIV-1 protease with T26S mutation.

18. The process according to claim 15, wherein:
(i) the pCMV-4070AAR, pCMV-GaLV10A1AR and pCMV-RD114AAR plasmids encode 4070A, GaLV10A1 and RD114A, respectively, with the deletion of the R-peptide from the cytoplasmic tail, being each of these plasmids amplified by inverse PCR from the parental plasmids, to remove the nucleotides coding for the R-peptide of the cytoplasmic tail of the envelope glycoprotein genes,
and
(ii) the pCMV-4070Agiflet, pCMV-GaLV10A1giflet and pCMV-RD114Agiflet encode 4070A, GaLV10A1 and RD114A, respectively, with a synthetic R-peptide cleavage site sequence reported as the most efficiently cleaved peptide site GSGIF↓LETSL by the HIV-1 protease being the construction of these plasmids conducted in two steps to replace the natural R-peptide cleavage site sequence with the synthetic sequence, where in the first step, each plasmid was amplified by inverse PCR from the parental plasmids to substitute the sequence VLTQ of the natural R-peptide cleavage site with the LETSL, and in the second step, the same approach was used to replace the VQAL sequence with the GSGIF sequence.

19. A cargo delivery carrier comprising the viral vector according to claim 12.

20. A cargo delivery carrier comprising a viral vector generated from the cell according to claim 13.

21. A cargo delivery carrier comprising a viral vector generated from the cell according to claim 14.

22. A composition comprising the cargo delivery carrier according to claim 19.

23. A composition comprising the cargo delivery carrier according to claim 20.

24. A composition comprising the cargo delivery carrier according to claim 21.

25. A method of using the modified envelope glycoprotein according to claim 11 for enhancing viral titers the R peptide region and/or cleavage site of the HIV-1 protease in the TM subunit.
